(19) United States
(12) Patent Application Publication
(10) Pub. No.: US 2003/0109519 A1
(43) Pub. Date: Jun. 12, 2003

(54) Use of selective potassium channel openers

(76) Inventor: Jeppe Sturis, Vaerlose (DK)

Correspondence Address: Reza Green, Esq., Novo Nordisk Pharmaceuticals, Inc., 100 College Road West, Princeton, NJ, 08540, US

(21) Appl. No.: 10/303,274

(22) Filed: Nov. 25, 2002

Related U.S. Application Data
(60) Provisional application No. 60/337,019, filed on Dec. 5, 2001.

(30) Foreign Application Priority Data
Nov. 30, 2001 (DK) PA 2001 01773

(51) Int. Cl. A61K 31/549

(52) U.S. Cl. 514/223.2

(57) ABSTRACT

The present invention relates to a use of SUR1/Kir6.2 selective potassium channel openers for the preparation of a pharmaceutical composition for the prevention or the treatment of diabetes in women with prior Gestational Diabetes Mellitus (GDM) as well as a pharmaceutical composition for use in the treatment of diabetes in women with prior GDM.

### FIELD OF INVENTION

The present invention relates to a use of SUR1/Kir6.2 selective potassium channel openers for the preparation of a medicament for the prevention or the treatment of diabetes in women with prior Gestational Diabetes Mellitus (GDM) as well as a pharmaceutical composition for use in the treatment of diabetes in women with prior GDM.

### BACKGROUND OF THE INVENTION

Potassium channels play an important role in the physiological and pharmacological control of cellular membrane potential. Amongst the different types of potassium channels are the ATP-sensitive (K_{ATP}-) channels, which are regulated by changes in the intracellular concentration of adenosine triphosphate. The K_{ATP}-channels have been found in cells from various tissues such as cardiac cells, pancreatic cells, skeletal muscles, smooth muscles, central neurons and adenohypophysis cells. The channels have been associated with diverse cellular functions for example hormone secretion (insulin from pancreatic beta cells, growth hormone and prolactin from adenohypophysis cells), vasodilation (in smooth muscle cells), cardiac action potential duration, neurotransmitter release in the central nervous system.

Modulators of the K_{ATP}-channels have been found to be of importance for the treatment of various diseases. Certain sulphonylureas, which have been used for the treatment of non-insulin-dependent diabetes mellitus, act by stimulating insulin release through an inhibition of the K_{ATP}-channels on pancreatic beta-cells.

The potassium channel openers (PCOs), which comprise a heterogeneous group of compounds, have been found to be able to relax vascular smooth muscles and have therefore been used for the treatment of hypertension.

Potassium channel openers hyperpolarize neurons and inhibit neurotransmitter release and it is expected that PCOs can be used for the treatment of various diseases of the central nervous system, e.g. epilepsia, ischemia and neurodegenerative diseases, such as Alzheimer's disease, and for the management of pain.

It has been shown that diazoxide (7-chloro-3-methyl-2H-1,2,4-benzothiadiazine 1,1-dioxide) and certain 3-(alkylamino)-4H-pyrido[4,3-e]-1,2,4-thiadiazine 1,1-dioxide derivatives inhibit insulin release by an activation of K_{ATP}-channels on pancreatic beta-cells (Pirotte B. et al. Biochem. Pharmacol, 47, 1381-1386 (1994); Pirotte B. et al., J. Med. Chem., 36, 3211-3213 (1993)).

Recently it has been shown that women with former Gestational Diabetes Mellitus (GDM) have a reduced conversion rate to diabetes when treated with troglitazone (Buchanan T A et al., 2001, Diabetes 50 (suppl 2):A81. The observed effect was greatest for those women in whom troglitazone therapy resulted in the greatest reduction in insulin secretion after an intravenous glucose tolerance test. It has also been suggested that treatment of with diazoxide (a non-selective potassium channel opener) may work in the same way as troglitazone in “resting” the beta-cell (Buchanan T A et al., 2001, JCEM 86: 989-993).

Normally an increase in the blood sugar level results in insulin secretion by the pancreatic β-cells. This is the result of an increase in the intracellular ATP/ADP ratio, causing ATP-sensitive K⁺ channels to close, which depolarizes the plasma membrane and promotes Ca²⁺ influx leading to insulin release. A low blood sugar level on the other hand will decrease the intracellular ATP/ADP ratio, causing ATP-sensitive K⁺ channels to open, which hyperpolarizes the plasma membrane and inhibits Ca²⁺ influx, preventing insulin release. Insulin release leads to a decrease in the amount of glucose in the blood by promoting glucose uptake by cells and increasing the capacity of the liver to synthesize glucogen. Therefore a reduction in the release of insulin normally would lead to an increase in blood sugar levels and thus a decrease in glucose tolerance.

It is known that SUR1/Kir6.2 channels are involved in the release of insulin as described above and that potassium channel openers therefore will affect release of insulin. However, it has now surprisingly been found that treatment with SUR1/Kir6.2 selective potassium channel openers at the same time can reduce hyperinsulinaemia without resulting in a deterioration of glucose tolerance.

### SUMMARY OF THE INVENTION

The present invention relates to a use of SUR1/Kir6.2 selective potassium channel openers for the preparation of a medicament for the treatment or the prevention of diabetes in women with prior GDM.

More specifically, the present invention relates to the use of compounds of the general formula (I):

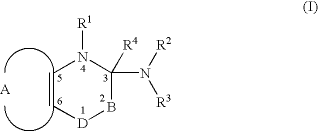

wherein

B represents >NR⁵ or >CR⁵R⁶, wherein R⁵ and R⁶ independently are hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen; or R⁵ and R⁴ together represent one of the bonds in a double bond between the atoms 2 and 3 of formula (I);

D represents —S(═O)₂— or —S(═O)—; or

D-B represents —S(═O)(R⁷)═N—

wherein R⁷ is C₁₋₆-alkyl; or aryl or heteroaryl optionally mono- or poly substituted with halogen, hydroxy, C₁₋₆-alkoxy, aryloxy, arylalkoxy, nitro, amino, C₁₋₆-monoalkyl- or dialkylamino, cyano, acyl, or C₁₋₆-alkoxycarbonyl;

R¹ is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);

R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen

R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₋₆-alkyl;

wherein R⁸ is hydrogen; C₃₋₆-cycloalkyl or (C₃₋₆-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen, oxygen or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;

X is O or S;

R⁹ is hydrogen; C₁₋₆-alkyl; C₂₋₆-alkenyl; C₃₋₆-cycloalkyl optionally mono- or poly substituted with C₁₋₆-alkyl, halogen, hydroxy or C₁₋₆-alkoxy; or R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or

R³ is

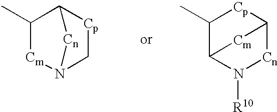

wherein n, m, p independently are 0, 1, 2, 3 and R¹¹ is hydrogen; hydroxy; C₁₋₆-alkoxy; C₃₋₆-cycloalkyl optionally mono- or poly substituted with C₁₋₆-alkyl, halogen, hydroxy or C₁₋₆-alkoxy; C₁₋₆-alkyl, C₂₋₆-alkenyl or C₂₋₆-alkynyl optionally mono- or poly substituted with halogen; or

R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo; A together with carbon atoms 5 and 6 of formula (I) represents a 5 or 6 membered heterocyclic system comprising one or more nitrogen-, oxygen- or sulfur atoms, the heterocyclic systems optionally being mono- or poly substituted with halogen; C₁₋₁₂-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl-methyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl-amino; C₁₋₆-monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)-C₁₋₆-alkyl the oxadiazolyl group optionally being substituted with C₁₋₆-alkyl or C₃₋₆-cycloalkyl; or a 5-6 membered nitrogen containing ring, optionally substituted with phenyl or C₁₋₆-alkyl; or a pharmaceutically acceptable salt thereof, for the preparation of a pharmaceutical composition for the treatment or the prevention of diabetes in women with prior Gestational Diabetes Mellitus (GDM).

In a further aspect the present invention relates to a pharmaceutical composition for use in the treatment or prevention of diabetes in women with prior GDM, comprising a compound of the formula (I) or (Ia) or a pharmaceutically acceptable salt thereof with a pharmaceutically acceptable acid or base, or any optical isomer or mixture of optical isomers including a racemic mixture, or any tautomeric form together with one or more pharmaceutically acceptable carriers or diluents.

In a still further aspect the present invention relates to a method for treating or preventing diabetes in women with prior GDM comprising administering an effective amount of a compound of the formula (I) or (Ia) to said subject.

### BRIEF DESCRIPTION OF DRAWINGS

FIG. 1 shows an oral glucose tolerance test in panel A and perfused pancreas in panel B.

### DEFINITIONS

Prior to a discussion of the detailed embodiments of the invention, a definition of specific terms related to the main aspects of the invention is provided.

The following is a detailed definition of the terms used to describe the compounds of the invention.

The term “prevention” in the context of “the treatment or the prevention of diabetes” means that the development of diabetes in women with prior GDM can be delayed or attenuated. Women with prior GDM have an increased risk of developing diabetes but the onset of the disease can be delayed and/or the severity of the disease attenuated by administration of a compound according to the present invention.

The term “halogen” designates an atom selected from the group consisting of F, Cl, Br and I.

The terms “C₁₋₆-alkyl”, “C₁₋₁₂-alkyl” and “C₁₋₁₈-alkyl” as used herein, alone or in combination, designates a straight or branched, saturated hydrocarbon chain having the indicated number of carbon atoms. Representatives examples include, but are not limited to methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-hexyl, 1,2-dimethylpropyl, 2,2-dimethylpropyl, 1,2,2-trimethylpropyl and the like. The term “C₁₋₁₈-alkyl” as used herein also includes secondary C₃₋₆-alkyl and tertiary C₄₋₆-alkyl.

The term “C₁₋₆-alkoxy” as used herein, alone or in combination, refers to a straight or branched monovalent substituent comprising a C₁₋₆-alkyl group linked through an ether oxygen having its free valence bond from the ether oxygen and having 1 to 6 carbon atoms. Representatives groups include, but are not limited to methoxy, ethoxy, propoxy, isopropoxy, butoxy, isobutoxy, sec-butoxy, tert-butoxy, n-pentoxy, neopentoxy, tert-pentoxy, n-hexoxy, isohexoxy and the like.

The term “C₂₋₆-alkenyl” as used herein refers to a straight or branched, unsaturated hydrocarbon chain having 2-6 carbon atoms and one double bond. Examples of such groups include, but are not limited to vinyl, 1-propenyl, 2-propenyl, allyl, isopropenyl, n-butenyl, n-pentenyl, n-hexenyl and the like.

The term “C₂₋₆-alkynyl” as used herein refers to a straight or branched, unsaturated hydrocarbons which contain triple bonds. Examples of such groups include, but are not limited to —C≡CH, —C≡CCH₃, —CH₂C—CH, —CH₂CH₂C═CH, —CH(CH₃)C—CH and the like.

The term “C₁₋₆-alkylthio” as used herein, alone or in combination, refers to a straight or branched monovalent substituent comprising a lower alkyl group linked through a divalent sulfur atom having its free valence bond from the sulfur atom and having 1 to 6 carbon atoms. Representative examples include, but are not limited to, methylthio, ethylthio, n-propylthio, isopropylthio, butylthio, isobutylthio, sec-butylthio, tert-butylthio, n-pentylthio, isopentylthio, neopentylthio, tert-pentylthio, n-hexylthio, isohexyl and the like.

The term “C₃₋₆-cycloalkyl” as used herein refers to a radical of a saturated cyclic hydrocarbon with the indicated number of carbons. Representative examples are cyclopropyl, cyclobutyl, cyclopentyl, cyclohexyl, cycloheptyl, cyclooctyl and the like.

The term “C₁₋₆-alkoxy-C₁₋₆-alkyl” as used herein refers to a group of 2-12 carbon atoms interrupted by an O. Representative examples are CH₂—O—CH₃, CH₂—O—CH₂—CH₃, CH₂—O—CH(CH₃)₂ and the like.

The term “perhalomethyl” means trifluoromethyl, trichloromethyl, tribromomethyl or triiodomethyl.

The term “C₁₋₆-monoalkylamino” as used herein refers to an amino group wherein one of the hydrogen atoms is substituted with a straight or branched, saturated hydrocarbon chain having the indicated number of carbon atoms such as e.g. methylamino, ethylamino, propylamino, n-butylamino, sec-butylamino, isobutylamino, tert-butylamino, n-pentylamino, 2-methylbutylamino, n-hexylamino, 4-methylpentylamino, neopentylamino, n-hexylamino, 2,2-dimethylpropylamino and the like.

The term “C₁₋₆-dialkylamino” as used herein refers to an amino group wherein the two hydrogen atoms independently are substituted with a straight or branched, saturated hydrocarbon chain having the indicated number of carbon atoms; such as dimethylamino, N-ethyl-N-methylamino, diethylamino, dipropylamino, N-(n-butyl)-N-methylamino, di(n-pentyl)amino, and the like.

The term “acyl” as used herein refers to a monovalent substituent comprising a C₁₋₆-alkyl group linked through a carbonyl group; such as e.g. acetyl, propionyl, butyryl, isobutyryl, pivaloyl, valeryl, and the like.

The term “C₁₋₆-alkoxycarbonyl” as used herein refers to a monovalent substituent comprising a C₁₋₆-alkoxy group linked through a carbonyl group; such as e.g. methoxycarbonyl, carbethoxy, propoxycarbonyl, isopropoxycarbonyl, n-butoxycarbonyl, sec-butoxycarbonyl, tert-butoxycarbonyl, 3-methylbutoxycarbonyl, n-hexoxycarbonyl and the like.

The term “3-12 membered mono- or bicyclic system” as used herein refers to a monovalent substituent of formula —NR²R³ or —NR⁸R⁹ where R² and R³, or R⁸ and R⁹ together with the nitrogen atom form a 3-12 membered mono- or bicyclic system, in which one or more of the carbon atoms may be exchanged with nitrogen, oxygen or sulfur, such as 1-pyrrolidyl, piperidino, morpholino, thiomorpholino, 4-methylpiperazin-1-yl, 7-azabicyclo[2.2.1]heptan-7-yl, tropanyl and the like.

The term “3-6 membered saturated ring system” as used herein refers to a monovalent substituent comprising a monocyclic saturated system containing one or more hetero atoms selected from nitrogen, oxygen and sulfur and having 3-6 members and having its free valence from a carbon atom, e.g. 2-pyrrolidyl, 4-piperidyl, 3-morpholinyl, 1,4-dioxan-2-yl, 5-oxazolidinyl, 4-isoxazolidinyl or 2-thiomorpholinyl.

The term “bicycloalkyl” as used herein refers to a monovalent substituent comprising a bicyclic structure made of 6-12 carbon atoms such as e.g. 2-norbornyl, 7-norbornyl, 2-bicyclo[2.2.2]octyl and 9-bicyclo[3.3.1]nonanyl.

The term “aryl” as used herein refers to phenyl, 1-naphthyl or 2-naphthyl.

The term “heteroaryl” as used herein, alone or in combination, refers to a monovalent substituent comprising a 5-6 membered monocyclic aromatic system or a 9-10 membered bicyclic aromatic system containing one or more heteroatoms selected from nitrogen, oxygen and sulfur, e.g. pyrrole, imidazole, pyrazole, triazole, pyridine, pyrazine, pyrimidine, pyridazine, isothiazole, isoxazole, oxazole, oxadiazole, thiadiazole, quinoline, isoquinoline, quinazoline, quinoxaline, indole, benzimidazole, benzofuran, pteridine and purine.

The term “arylalkyl” as used herein refers to a straight or branched saturated carbon chain containing from 1 to 6 carbons substituted with an aromatic carbohydride; such as benzyl, phenethyl, 3-phenylpropyl, 1-naphtylmethyl, 2-(1-naphtyl)ethyl and the like.

The term “aryloxy” as used herein refers to phenoxy, 1-naphthyloxy or 2-naphthyloxy.

The term “arylalkoxy” as used herein refers to a C₁₋₆-alkoxy group substituted with an aromatic carbohydride, such as benzyloxy, phenethoxy, 3-phenylpropoxy, 1-naphthylmethoxy, 2-(1-naphtyl)ethoxy and the like.

The term “heteroarylalkyl” as used herein refers to a straight or branched saturated carbon chain containing from 1 to 6 carbons substituted with a heteroaryl group; such as (2-furyl) methyl, (3-furyl)methyl, (2-thienyl)methyl, (3-thienyl)methyl, (2-pyridyl)methyl, 1-methyl-1-(2-pyrimidyl)ethyl and the like.

The term “C₁₋₆-alkylsulfonyl” as used herein refers to a monovalent substituent comprising a C₁₋₆-alkyl group linked through a sulfonyl group such as e.g. methylsulfonyl, ethylsulfonyl, n-propylsulfonyl, isopropylsulfonyl, n-butylsulfonyl, sec-butylsulfonyl, iso-butylsulfonyl, tert-butylsulfonyl, n-pentylsulfonyl, 2-methylbutylsulfonyl, 3-methylbutylsulfonyl, n-hexylsulfonyl, 4-methylpentylsulfonyl, neopentylsulfonyl, n-hexylsulfonyl and 2,2-dimethylpropylsulfonyl.

The term “C₁₋₆-monoalkylaminosulfonyl” as used herein refers to a monovalent substituent comprising a C₁₋₆-monoalkylamino group linked through a sulfonyl group such as e.g. methylaminosulfonyl, ethylaminosulfonyl, n-propylaminosulfonyl, isopropylaminosulfonyl, n-butylaminosulfonyl, sec-butylaminosulfonyl, iso-butylaminosulfonyl, tert-butylaminosulfonyl, n-pentylaminosulfonyl, 2-methylbutylaminosulfonyl, 3-methylbutylaminosulfonyl, n-hexylaminosulfonyl, 4-methylpentylaminosulfonyl, neopentylaminosulfonyl, n-hexylaminosulfonyl and 2,2-dimethylpropylaminosulfonyl.

The term “C₁₋₆-dialkylaminosulfonyl” as used herein refers to a monovalent substituent comprising a C₁₋₆-dialkylamino group linked through a sulfonyl group such as dimethylaminosulfonyl, N-ethyl-N-methylaminosulfonyl, diethylaminosulfonyl, dipropylaminosulfonyl, N-(n-butyl)-N-methylaminosulfonyl, di(n-pentyl)aminosulfonyl, and the like.

The term “C₁₋₆-alkylsulfinyl” as used herein refers to a monovalent substituent comprising a straight or branched C₁₋₆-alkyl group linked through a sulfinyl group (—S(═O)—); such as e.g. methylsulfinyl, ethylsulfinyl, isopropylsulfinyl, butylsulfinyl, pentylsulfinyl, and the like.

The term “C₁₋₆-alkylcarbonylamino” as used herein refers to an amino group wherein one of the hydrogen atoms is substituted with an acyl group, such as e.g. acetamido, propionamido, isopropylcarbonylamino, and the like.

The term “(C₃₋₆-cycloalkyl)C₁₋₆-alkyl” as used herein, alone or in combination, refers to a straight or branched, saturated hydrocarbon chain having 1 to 6 carbon atoms and being monosubstituted with a C₃₋₆-cycloalkyl group, the cycloalkyl group optionally being mono- or polysubstituted with C₁₋₆-alkyl, halogen, hydroxy or C₁₋₆-alkoxy; such as e.g. cyclopropylmethyl, (1-methylcyclopropyl)methyl, 1-(cyclopropyl)ethyl, cyclopentylmethyl, cyclohexylmethyl, and the like.

The term “arylthio” as used herein, alone or in combination, refers to an aryl group linked through a divalent sulfur atom having its free valence bond from the sulfur atom, the aryl group optionally being mono- or polysubstituted with C₁₋₆-alkyl, halogen, hydroxy or C₁₋₆alkoxy; e.g. phenylthio, (4-methylphenyl)-thio, (2-chlorophenyl) thio, and the like.

The term “arylsulfinyl” as used herein refers to an aryl group linked through a sulfinyl group (—S(═O)-), the aryl group optionally being mono- or polysubstituted with C₁₋₆-alkyl, halogen, hydroxy or C₁₋₆-alkoxy; such as e.g. phenylsulfinyl, (4-chlorophenyl)sulfinyl, and the like.

The term “arylsulfonyl” as used herein refers to an aryl group linked through a sulfonyl group, the aryl group optionally being mono- or polysubstituted with C₁₋₆-alkyl, halogen, hydroxy or C₁₋₆-alkoxy; such as e.g. phenylsulfonyl, tosyl, and the like.

The term “C₁₋₆-monoalkylaminocarbonyl” as used herein refers to a monovalent substituent comprising a C₁₋₆-monoalkylamino group linked through a carbonyl group such as e.g. methylaminocarbonyl, ethylaminocarbonyl, n-propylaminocarbonyl, isopropylaminocarbonyl, n-butylaminocarbonyl, sec-butylaminocarbonyl, isobutylaminocarbonyl, tert-butylaminocarbonyl, n-pentylaminocarbonyl, 2-methylbutylaminocarbonyl, 3-methylbutylaminocarbonyl, n-hexylaminocarbonyl, 4-methylpentylaminocarbonyl, neo-pentylaminocarbonyl, n-hexylaminocarbonyl and 2-2-dimethylpropylaminocarbonyl.

The term “C₁-dialkylaminocarbonyl” as used herein refers to a monovalent substituent comprising a C₁₋₆-dialkylamino group linked through a carbonyl group such as dimethylaminocarbonyl, N-ethyl-N-methylaminocarbonyl, diethylaminocarbonyl, dipropylaminocarbonyl, N-(n-butyl)-N-methylaminocarbonyl, di(n-pentyl)aminocarbonyl, and the like.

The term “C₁₋₆-monoalkylaminocarbonylamino” as used herein refers to an amino group wherin one of the hydrogen atoms is substituted with a C₁₋₆-monoalkylaminocarbonyl group, e.g. methylaminocarbonylamino, ethylamino-carbonylamino, n-propylaminocarbonylamino, isopropylaminocarbonylamino, n-butylaminocarbonylamino, sec-butylam inocarbonylamino, isobutylaminocarbonylamino, tert-butylaminocarbonylamino, and 2-methylbutylaminocarbonylamino.

The term “C₁₋₆-dialkylaminocarbonylamino” as used herein refers to an amino group wherein one of the hydrogen atoms is substituted with a C₁₋₆-dialkylaminocarbonyl group, such as dimethylaminocarbonylamino, N-ethyl-N-methylaminocarbonylamino, diethylaminocarbonylamino, dipropylaminocarbonylamino, N-(n-butyl)-N-methylaminocarbonylamino, di(n-pentyl) aminocarbonylamino, and the like.

The term “5- or 6-membered heterocyclic system” as used herein refers to: a monocyclic unsaturated or saturated system containing one, two or three hetero atoms selected from nitrogen, oxygen and sulfur and having 5 members, e.g. pyrrole, furan, thiophene, pyrroline, dihydrofuran, dihydrothiophene, imidazole, imidazoline, pyrazole, pyrazoline, oxazole, thiazole, isoxazole, isothiazole, 1,2,3-oxadiazole, furazan, 1,2,3-triazole, 1,2,3-thiadiazole or 2,1,3-thiadiazole; an aromatic monocyclic system containing one or more nitrogen atoms and having 6 members, e.g. pyridine, pyrazine, pyrimidine, pyridazine, 1,2,4-triazine, 1,2,3-triazine or tetrazine; a non-aromatic monocyclic system containing one or more hetero atoms selected from nitrogen, oxygen and sulfur and having 6 members, e.g. pyran, thiopyran, piperidine, dioxane, oxazine, isoxazine, dithiane, oxathine, thiazine, piperazine, thiadiazine, dithiazine or oxadiazine.

The term “5- or 6-membered nitrogen containing ring” as used herein refers to a monovalent substituent comprising a monocyclic unsaturated or saturated system containing one or more nitrogen atoms and having 5 or 6 members, e.g. pyrrolidinyl, pyrrolinyl, imidazolidinyl, pyrazolidinyl, pyrazolinyl, piperidyl, piperazinyl, pyrrolyl, 2H-pyrrolyl, imidazolyl, pyrazolyl, triazolyl, pyridyl, pyrazinyl, pyrimidinyl, pyridazinyl, morpholino, thiomorpholino, isothiazolyl, isoxazolyl, oxazolyl, oxadiazolyl, thiadiazolyl, 1,3-dioxolanyl and 1,4-dioxolanyl.

The term “4- to 12-membered bicyclic or tricyclic carbocyclic system” as used herein refers to a a monovalent substituent comprising a bicyclic or a tricyclic structure made of 4-12 carbon atoms such as e.g. bicyclo[2.1.1]hexane, bicyclo[2.2.1]heptane, bicyclo[2.2.2]octane, octahydrovpentalene, bicyclo[2.2.0]hexane, adamantane, noradamantane or tricyclo-(4.3.1.1 (3,8))undecane.

The term “treatment” as used herein is defined as the management and care of a patient for the purpose of combating the disease, condition, or disorder and includes the administration of the active compounds to prevent the onset of the symptoms or complications, or alleviating the symptoms or complications, or eliminating the disease, condition, or disorder.

### DETAILED DESCRIPTION OF THE INVENTION

It is known that SUR1/Kir6.2 channels are involved in the release of insulin as described above and that potassium channel opener therefore will affect release of insulin. However, it has now surprisingly been found that treatment with SUR1/Kir6.2 selective potassium channel openers at the same time can reduce hyperinsulinaemia without resulting in a deterioration of glucose tolerance as shown in example 1. The result of such treatment is a workload reduction (“resting”) of pancreatic 13-cells which can prevent or delay the onset of Type 2 diabetes in women who have previously had GDM.

In one embodiment, the present invention therefore relates to a use of SUR1/Kir6.2 selective potassium channel openers for the preparation of a pharmaceutical composition for the treatment or the prevention of diabetes in women with (prior) GDM.

Examples of such potassium channel agonists are compounds, which activate K_{ATP}-channels of the β-cell type (SUR1/Kir6.2).

Potassium channel agonists can readily be determined by those skilled in the art. Methods therefore has been described in e.g. WO 97/26264, WO 97/26265, WO 99/03861, WO 00/37474, and recently reviewed: McClenaghan: Diabetes, Obesitas and Metabolism, 1, 137-150, (1999); Yokoshiki: Am. J. Physiol. 0.274. C25-C37, (1998); Aguliar-Bryan: Endocrine Reviews, 20, 101-135, (1999).

In a further embodiment the present invention relates to the use of compounds of the general formula (I):

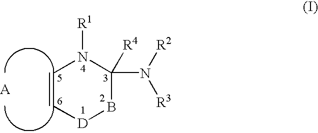

wherein

B represents >NR⁵ or >CR⁵R⁶, wherein R⁵ and R⁶ independently are hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen; or R⁵ and R⁴ together represent one of the bonds in a double bond between the atoms 2 and 3 of formula (I);

D represents —S(═O)₂— or —S(═O)—; or

D-B represents —S(═O)(R⁷)═N—

wherein R⁷ is C₁₋₆-alkyl; or aryl or heteroaryl optionally mono- or poly substituted with halogen, hydroxy, C₁₋₆-alkoxy, aryloxy, arylalkoxy, nitro, amino, C₁₋₆-monoalkyl- or dialkylamino, cyano, acyl, or C₁₋₆-alkoxycarbonyl;

R¹ is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);

R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen;

R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₋₆-alkyl;

wherein R⁸ is hydrogen; C₃₋₆-cycloalkyl or (C₃₋₆-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen-, oxygen- or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;

X is O or S;

R⁹ is hydrogen; C₁₋₆-alkyl; C₂₋₆-alkenyl; C₃₋₆-cycloalkyl optionally mono- or poly substituted with C₁₋₆-alkyl, halogen, hydroxy or C₁₋₆-alkoxy; or

R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or

R³ is

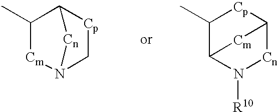

wherein n, m, p independently are 0, 1, 2, 3 and R¹⁰ is hydrogen; hydroxy; C₁₋₆-alkoxy; C₃₋₆-cycloalkyl optionally mono- or poly substituted with C₁₋₆-alkyl, halogen, hydroxy or C₁₋₆-alkoxy; C₁₋₆-alkyl, C₂₋₆-alkenyl or C₂₋₆-alkynyl optionally mono- or poly substituted with halogen; or

R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;

A together with carbon atoms 5 and 6 of formula (I) represents a 5 or 6 membered heterocyclic system comprising one or more nitrogen-, oxygen- or sulfur atoms, the heterocyclic systems optionally being mono- or poly substituted with halogen; C₁₋₁₂-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-Monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl-methyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl-amino; C₁₋₆-monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)-C₁₋₆-alkyl the oxadiazolyl group optionally being substituted with C₁₋₆-alkyl or C₃₋₆-cycloalkyl; or a 5-6 membered nitrogen containing ring, optionally substituted with phenyl or C₁₋₆-alkyl; or a pharmaceutically acceptable salt thereof, for the preparation of a pharmaceutical composition for the treatment or the prevention of diabetes in women with prior GDM.

Within its scope the invention includes all optical isomers of compounds of the present invention, some of which are optically active, and also their mixtures including racemic mixture thereof.

The scope of the invention also includes all tautomeric forms of the compounds of the present invention as well as metabolites or prodrugs.

A “metabolite” of a compound disclosed in this application is an active derivative of a compound disclosed herein which is produced when the compound is metabolized. Metabolites of compounds disclosed herein can be identified either by administration of a compound to a host and an analysis of blood samples from the host, or by incubation of compounds with hepatic cells in vitro and analysis of the incubant.

A “prodrug” is a compound that either is converted into a compound disclosed in the application in vivo or has the same active metabolite as a compound disclosed in this application.

The salts include pharmaceutically acceptable acid addition salts, pharmaceutically acceptable metal salts or optionally alkylated ammonium salts, such as hydrochloric, hydrobromic, hydroiodic, phosphoric, sulfuric, trifluoroacetic, trichloroacetic, oxalic, maleic, pyruvic, malonic, succinic, citric, tartaric, fumaric, mandelic, benzoic, cinnamic, methane-sulfonic, ethane sulfonic, picric and the like, and include acids related to the pharmaceutically acceptable salts listed in Journal of Pharmaceutical Science, 66, 2 (1977) and incorporated herein by reference, or lithium, sodium, potassium, magnesium and the like.

In another embodiment of the invention B of formula (I) is >NR⁵ and R⁵ and R⁴ together represent one of the bonds in a double bond between the atoms 2 and 3 of formula (I).

In another embodiment of the invention D is —S(═O)₂—.

In another embodiment of the invention R² is hydrogen or C₁₋₆-alkyl.

In another embodiment of the invention R³ is R⁸, OR⁸, NR⁸R⁹ or aryl, the aryl groups optionally being substituted with C₁₋₆-alkyl; wherein R⁸ is hydrogen; C₃₋₆-cycloalkyl; ₆-cycloalkyl)C₁₋₆-alkyl; a 3-6 membered saturated ring system comprising one, two or three nitrogen-, oxygen- or sulfur atoms; or straight or branched C₁₋₁₈-alkyl optionally substituted with halogen, hydroxy, C₁₋₆-alkoxy, C₁₋₆-alkylthio, C₃₋₆-cycloalkyl or aryl, R⁹ is hydrogen, C₁₋₆-alkyl or C₃₋₆-cycloalkyl; or R⁸ and R⁹ together with the nitrogen atom form a 4-6 membered ring.

In another embodiment of the invention wherein R³ is secondary C₃₋₆-alkyl, tertiary C₄₋₆-alkyl, C₃₋₆-cycloalkyl or (C₃₋₆-cycloalkyl)methyl.

In another embodiment of the invention A together with carbon atoms 5 and 6 of formula (I) forms a 5 membered heterocyclic system containing one hetero atom selected from nitrogen and sulfur, the heterocyclic system optionally being mono- or disubstituted with halogen; C₁₋₁₂-alkyl; C₃₋₆-cycloalkyl; cyano; cyanomethyl; perhalomethyl; sulfamoyl; C₁₋₆-alkylthio; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl or a 5-6 membered nitrogen containing ring, optionally substituted with phenyl or C₁₋₆-alkyl.

In another embodiment of the invention A together with carbon atoms 5 and 6 of formula (I) forms a 5 membered heterocyclic system containing two hetero atoms selected from nitrogen, oxygen and sulfur, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

In another embodiment of the invention A together with carbon atoms 5 and 6 of formula (I) forms a 6 membered aromatic heterocyclic system containing one, two or three nitrogen atoms, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylthio; C₁₋₆-alkylsulfonyl; C₁₋₆-alkylsulfinyl; arylthio, arylsulfinyl, arylsulfonyl, the aryll group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

Examples of specific compounds of formula (I) to be used according to this invention are: 6-Chloro-3-(1,2-dimethylpropyl)amino-4H-thieno[3,2-e]-1,2,4-thiadiazine 1,1-dioxide; 6-Chloro-3-ethylamino-4H-thieno[3,2-e]-1,2,4-thiadiazine 1,1-dioxide; 6-Chloro-3-isopropylamino-4H-thieno[3,2-e]-1,2,4-thiadiazine 1,1-dioxide; (R)-6-Chloro-3-(1-phenylethyl)amino-4H-thieno[3,2-e]-1,2,4-thiadiazine 1,1-dioxide; 3-Allylamino-6-chloro-4H-thieno[3,2-e]-1,2,4-thiadiazine 1,1-dioxide; 6-Chloro-3-cyclopropylamino-4H-thieno[3,2-e]-1,2,4-thiadiazine 1,1-dioxide; 6-Chloro-3-hexylamino-4H-thieno[3,2-e]-1,2,4-thiadiazine 1,1-dioxide; 6-Chloro-3-tetradecylamino-4H-thieno[3,2-e]-1,2,4-thiadiazine 1,1-dioxide; 6-Chloro-3-methylamino-4H-thieno[3,2,e]-1,2,4-thiadiazine 1,1-dioxide; 3-Benzylamino-6-chloro-4H-thieno[3,2,e]-1,2,4-thiadiazine 1,1-dioxide; 6-Chloro-3-octylamino-4H-thieno[3,2-e]-1,2,4-thiadiazine 1,1-dioxide; 6-Chloro-3-isobutylamino-4H-thieno[3,2-e]-1,2,4-thiadiazine 1,1-dioxide; 6-Chloro-3-(4-phenylbutyl)amino-4H-thieno[3,2-e]-1,2,4-thiadiazine 1,1-dioxide; 6-Chloro-3-(1,5-dimethyl hexyl)amino-4H-thieno[3,2-e]-1,2,4-thiadiazine 1,1-dioxide; 6-Chloro-3-propylamino-4H-thieno[3,2-e]-1,2,4-thiadiazine 1,1-dioxide; (R)-6-Chloro-3-(2-hydroxy-1-methylethyl) amino-4H-thieno[3,2-e]-1,2,4-thiadiazine 1,1-dioxide; (S)-6-Chloro-3-(2-hydroxy-1-methylethyl)amino-4H-thieno[3,2-e]-1,2,4-thiadiazine 1,1-dioxide; (R)-3-sec-Butylamino-6-chloro-4H-thieno[3,2-e]-1,2,4-thiadiazine 1,1-dioxide; 3-Butylamino-6-chloro-4H-thieno[3,2-e]-1,2,4-thiadiazine 1,1-dioxide; 3-Isopropylamino-7-methyl-4,7-dihydropyrazolo[4,3-e][1,2,4]thiadiazine 1,1-dioxide.

Another example of a specific compound of formula (I) to be used according to this invention is 6-Chloro-3-isopropylamino-4H-thieno[3,2-e]-1,2,4-thiadiazine 1,1-dioxide.

Other examples of specific compounds of formula (I) to be used according to this invention are: 3-Hydrazino-4H-pyrido[4,3-e]-1,2,4-thiadiazine 1,1-dioxide; 3-Benzylamino-4H-pyrido[4,3-e]-1,2,4-thiadiazine 1,1-dioxide; 3-(R)-(1-Phenylethylamino)-4H-pyrido[4,3-e]-1,2,4-thiadiazine 1,1-dioxide; 3-(S)-(1-Phenylethylamino)-4H-pyrido[4,3-e]-1,2,4-thiadiazine 1,1-dioxide; 3-Benzylamino-7-chloro-4H-pyrido[2,3-e]-1,2,4-thiadiazine 1,1-dioxide; 7-Chloro-3-(R)-(1-phenylethylamino)-4H-pyrido[2,3-e]-1,2,4-thiadiazine 1,1-dioxide; 7-Chloro-3-(S)-(1′-phenylethylamino)-4H-pyrido[2,3-e]-1,2,4-thiadiazine 1,1-dioxide; 3-Benzylamino-4H-pyrido[2,3-e]-1,2,4-thiadiazine 1,1-dioxide; 3-(R)-(1-Phenylethylamino)-4H-pyrido[2,3-e]-1,2,4-thiadiazine 1,1-dioxide; 3-(S)-(1-Phenylethylamino)-4H-pyrido[2,3-e]-1,2,4-thiadiazine 1,1-dioxide; 3-(Hexylamino)-4H-pyrido[4,3-e]-1,2,4-thiadiazine 1,1-dioxide; 7-Chloro-3-hexylamino-4H-pyrido[2,3-e]-1,2,4-thiadiazine 1,1-dioxide; 3-Octylamino-4H-pyrido[4,3-e]-1,2,4-thiadiazine 1,1-dioxide; 7-Chloro-3-octylamino-4H-pyrido [2,3-e]-1,2,4-thiadiazine 1,1-dioxide; 3-Allylamino-4H-pyrido[4,3-e]-1,2,4-thiadiazine 1,1-dioxide; 3-Allylamino-7-chloro-4H-pyrido[2,3-e]-1,2,4-thiadiazine 1,1-dioxide; 7-Chloro-3-(2-methoxy-1-methylethyl)amino-4H-pyrido[2,3-e]-1,2,4-thiadiazine 1,1-dioxide; 3-(2-Methoxy-1-methylethyl)amino-4H-pyrido[4, 3-e]-1,2,4-thiadiazine 1,1-dioxide; 3-(2-Hydroxy-1-methylethyl)amino-4H-pyrido[4,3-e]-1,2,4-thiadiazine 1,1-dioxide; 3-Benzylamino-2-methyl-2H-pyrido[4,3-e]-1,2,4-thiadiazine 1,1-dioxide; 2-Isopropylamino-3,3-dimethoxy-3H-pyrido [2,3-b][1,4]thiazine 4,4-dioxide.

Other examples of specific compounds of formula (I) to be used according to this invention are: 7-Cyano-3-isopropylamino-6-methyl-4H-thieno[2,3-e]-1,2,4-thiadiazine 1,1-dioxide; 7-Cyano-6-methyl-3-propylamino-4H-thieno[2,3-e]-1,2,4-thiadiazine 1,1-dioxide; 6-Chloro-3-isopentylamino-4H-thieno[3,2-e]-1,2,4-thiadiazine 1,1-dioxide; 6-Chloro-3-(1-methylheptyl)amino-4H-thieno[3,2-e]-1,2,4-thiadiazine 1,1-dioxide; 6-Chloro-3-(1-ethylpentyl)amino-4H-thieno[3,2-e]-1,2,4-thiadiazine 1,1-dioxide; 6-Chloro-3-(2-methylbutyl)-amino-4H-thieno[3,2-e]-1,2,4-thiadiazine, 1-dioxide; 6-Chloro-3-(1-methylhexyl)amino-4H-thieno[3,2-e]-1,2,4-thiadiazine 1,1-dioxide; 6-Chloro-3-cyclopentyl amino-4H-thieno[3,2-e]-1,2,4-thiadiazine 1,1-dioxide; 6-Chloro-3-cyclohexylmethylamino-4H-thieno[3,2-e]-1,2,4-thiadiazine 1,1-dioxide; Ethyl 3-(6-chloro-1,4-dihydro-1,1-dioxothieno[3,2-e]-1λ⁶,2,4-thiadiazin-3-ylamino)-butanoate; 3-(6-Chloro-1,4-dihydro-1,1-dioxothieno[3,2-e]-1λ⁶,2,4-thiadiazin-3-ylamino)butanoic acid; 6-Chloro-3-(3-hydroxy-1-methylpropyl)amino-4H-thieno[3,2-e]-1,2,4-thiadiazine 1,1-dioxide; (R)-6-Chloro-3-(1-phenyethyl)amino-4H-thieno[3,2-e]-1,2,4-thiadiazine 1,1-dioxide; (S)-3-sec-Butylamino-6-chloro-4H-thieno[3,2-e]-1,2,4-thiadiazine 1,1-dioxide; 6-Chloro-3-isopropylamino-4H-thieno[2,3-e]-1,2,4-thiadiazine 1,1-dioxide; 6-Chloro-3-cyclopentylamino-4H-thieno[2,3-e]-1,2,4-thiadiazine 1,1-dioxide; 6-Bromo-3-isopropylamino-4H-thieno[3,2-e]-1,2,4-thiadiazine 1,1-dioxide; 3-Isopropylamino-4H-thieno[3,2-e]-1,2,4-thiadiazine 1,1-dioxide; 6-Fluoro-3-isopropylamino-4H-thieno[3,2-e]-1,2,4-thiadiazine 1,1-dioxide; 3-Cyclobutylamino-5,6-dimethyl-4H-thieno[3,2-e]-1,2,4-thiadiazine 1,1-dioxide; 3-Cyclopentylamino-5,6-dimethyl-4H-thieno[3,2-e]-1,2,4-thiadiazine 1,1-dioxide; 3-Isopropylamino-6,7-dimethyl-4H-thieno[2,3-e]-1,2,4-thiadiazine 1,1-dioxide; 3-Cyclobutylamino-6,7-dimethyl-4H-thieno[2,3-e]-1,2,4-thiadiazine 1,1-dioxide; 3-Cyclopentylamino-6,7-dimethyl-4H-thieno[2,3-e]-1,2,4-thiadiazine 1,1-dioxide; 5-Chloro-3-isopropylamino-4H-thieno[3,2-e]-1,2,4-thiadiazine 1,1-dioxide; 5-Chloro-3-propylamino-4H-thieno[3,2-e]-1,2,4-thiadiazine 1,1-dioxide; 5-Chloro-3-cyclopentylamino-4H-thieno[3,2-e]-1,2,4-thiadiazine 1,1-dioxide; 5-Chloro-6-methyl-3-isopropylamino-4H-thieno[3,2-e]-1,2,4-thiadiazine 1,1-dioxide; 6-chloro-3-isopropylamino-5-methyl-4H-thieno[3,2-e]-1,2,4-thiadiazine 1,1-dioxide; 6-chloro-3-cyclopentylamino-5-methyl-4H-thieno[3,2-e]-1,2,4-thiadiazine 1,1-dioxide; 6-Fluoro-3-propylamino-4H-thieno[3,2-e]-1,2,4-thiadiazine 1,1-dioxide; 6-Fluoro-3-cyclopentylamino-4H-thieno[3,2-e]-1,2,4-thiadiazine 1,1-dioxide; 5-Fluoro-3-propylamino-4H-thieno[3,2-e]-1,2,4-thiadiazine 1,1-dioxide; 5-Fluoro-3-isopropylamino-4H-thieno[3,2-e]-1,2,4-thiadiazine 1,1-dioxide; 3-Isopropylamino-7-methyl-4H-thieno[2,3-e]-1,2,4-thiadiazine 1,1-dioxide; 6-Chloro-3-cyclobutylamino-4H-thieno[3,2-e]-1,2,4-thiadiazine 1,1-dioxide; 6-Chloro-3-(2-hydroxyethyl)amino-4H-thieno[3,2-e]-1,2,4-thiadiazine 1,1-dioxide; (±)-3-exo-Bicyclo[2.2.1]hept-2-ylamino-6-chloro-4H-thieno[3,2-e]-1,2,4-thiadiazine 1,1-dioxide; (R)-6-Chloro-3-(2-hydroxypropyl)amino-4H-thieno[3,2-e]-1,2,4-thiadiazine 1,1-dioxide; 6-Bromo-3-isopropylamino-4H-thieno[3,2-e]-1,2,4-thiadiazine 1,1-dioxide; 5,6-Dibromo-3-isopropylamino-4H-thieno[3,2-e]-1,2,4-thiadiazine 1,1-dioxide; 6-Chloro-3-cyclohexylamino-4H-thieno[3,2-e]-1,2,4-thiadiazine 1,1-dioxide; 6-Chloro-3-(furan-2-ylmethyl)amino-4H-thieno[3,2-e]-1,2,4-thiadiazine 1,1-dioxide; 6-Chloro-3-(1-ethylpropyl) amino-4H-thieno[3,2-e]-1,2,4-thiadiazine 1,1-dioxide; 6-Bromo-3-cyclopentylamino-4H-thieno[3,2-e]-1,2,4-thiadiazine 1,1-dioxide; 6-Chloro-3-(2-methylallyl)amino-4H-thieno[3,2-e]-1,2,4-thiadiazine 1,1-dioxide; 6-Cyano-3-isopropylamino-4H-thieno[3,2-e]-1,2,4-thiadiazine 1,1-dioxide.

In another embodiment of the invention the general formula (I) is selected from

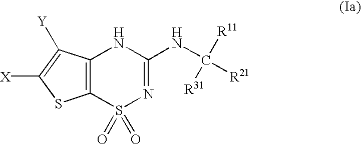

wherein

X and Y independently are hydrogen, halogen, perhalomethyl, C₁₋₆-alkyl or C₁₋₆-alkoxy;

R¹¹, R²¹ and R³¹ independently are C₁₋₆-alkyl, C₂₋₆-alkenyl, C₂₋₆-alkynyl, C₃₋₆-cycloalkyl, carboxy, C₁₋₆-alkoxycarbonyl or aryl, all of which are optionally being mono- or polysubstituted with halogen, hydroxy, oxo, or aryl; or

R¹¹ is as defined above and R²¹—C—R³¹ form a C₃₋₆-cycloalkyl group, optionally being mono- or polysubstituted with C₁₋₆-alkyl, perhalomethyl, halogen, hydroxy or aryl; or

—CR¹¹R²¹R³¹ form a 4- to 12-membered bicyclic or tricyclic carbocyclic system, optionally being mono- or polysubstituted with C₁₋₆-alkyl, perhalomethyl, halogen, hydroxy or aryl; or a salt thereof with a pharmaceutically acceptable acid or base including all optical isomers of compounds of formula (Ia).

In another embodiment of the invention, in formula (Ia) X is halogen and Y is hydrogen.

In another embodiment of the invention, in formula (Ia), X is chloro.

In another embodiment of the invention, in formula (Ia), R¹¹, R²¹ and R³¹ all are C₁₋₆-alkyl.

In another embodiment of the invention, in formula (Ia), R¹¹ is methyl.

In another embodiment of the invention, in formula (Ia), R²¹—C—R³¹ forms a C₃₋₆-cycloalkyl group.

In another embodiment of the invention, in formula (Ia), —CR¹¹R²¹R³¹ forms a tricyclic carbocyclic system.

Examples of specific compounds of formula (Ia) to be used according to this invention are: 3-tert-Butylamino-6-chloro-4H-thieno[3,2-e]-1,2,4-thiadiazine 1,1-dioxide; 6-Chloro-3-(1,1-dimethylpropylamino)-4H-thieno[3,2-e]-1,2,4-thiadiazine 1,1-dioxide; 6-Chloro-3-(1-methylcyclopropyl)amino-4H-thieno[3,2-e]-1,2,4-thiadiazine 1,1-dioxide; 6-Chloro-3-(2-hydroxy-1,1-dimethylethylamino)-4H-thieno[3,2-e]-1,2,4-thiadiazine 1,1-dioxide; 6-Chloro-3-(1,1,3,3-tetramethylbutylamino)-4H-thieno[3,2-e]-1,2,4-thiadiazine 1,1-dioxide; 3-(1-Adamantyl)amino-6-chloro-4H-thieno[3,2-e]-1,2,4-thiadiazine 1,1-dioxide; 1-(6-Chloro-1,4-dihydro-1,1-dioxo-thieno[3,2-e]-1λ⁶,2,4-thiadiazin-3-ylamino)-cyclopropanecarboxylic acid ethyl ester; 6-Chloro-3-(1-methyl-1-phenylethyl)amino-4H-thieno[3,2-e]-1,2,4-thiadiazine 1,1-dioxide; 6-Chloro-3-(1-hydroxymethylcyclopentyl)amino-4H-thieno[3,2-e]-1,2,4-thiadiazine 1,1-dioxide; 1-(6-Chloro-1,4-dihydro-1,1-dioxo-thieno[3,2-e]-1λ⁶,2,4-thiadiazin-3-ylamino)-cyclopropanecarboxylic acid; 6-Chloro-3-(1-methylcyclobutyl)amino-4H-thieno[3,2-e]-1,2,4-thiadiazine 1,1-dioxide; 6-Chloro-3-(1-methylcyclohexyl)amino-4H-thieno[3,2-e]-1,2,4-thiadiazine 1,1-dioxide; 6-Chloro-3-(1-methylcyclopentyl)amino-4H-thieno[3,2-e]-1,2,4-thiadiazine 1,1-dioxide; 6-Chloro-3-(1-ethylcyclobutyl)amino-4H-thieno[3,2-e]-1,2,4-thiadiazine 1,1-dioxide.

Another example of a specific compound of formula (Ia) to be used according to this invention is 6-Chloro-3-(1-methylcyclopropyl)amino-4H-thieno[3,2-e]-1,2,4-thiadiazine 1,1-dioxide.

The compounds of formula (I) and (Ia) of the present invention may be prepared by using the methods taught in e.g. WO 97/26264, WO 97/26265, WO 99/03861 and WO 00/37474, which are hereby incorporated by reference.

In addition the compounds of the present invention may be used in combination with compounds that are used for the treatment of type 2 diabetes, obesitas or hypertension.

In such embodiments, the pharmaceutical composition of the invention may comprise a compound of formula (I) or (Ia) combined with one or more other pharmacologically active compounds, e.g. an antidiabetic or other pharmacologically active material. Suitable antidiabetics comprise short and long acting insulins, insulin analogues, insulin sensitizers, insulin secretagogues as well as orally active hypoglycaemic agents such as sulphonylureas, e.g. glibenclamide and glipizide; biguanides, e.g. metformin; benzoic acid derivatives, e.g. repaglinide; thiazolidinediones, e.g. rosiglitazone, pioglitazone and ciglitazone; glucagon like peptide 1 (GLP-1), GLP-1 derivatives and GLP-1 analogues; peroxisome proliferating activated receptor (PPAR) ligands including the PPAR-alpha, PPAR-gamma and PPAR-delta subtypes; inhibitors of α-glucosidase, e.g. acarbose and voglibose, inhibitors of hepatic enzymes responsible for the biosynthesis of glucose, e.g. glycogen phosphorylase inhibitors.

Pharmaceutical Compositions

The present invention also relates to pharmaceutical compositions comprising, as an active ingredient, at least one of the compounds of the present invention or a pharmaceutically acceptable salt thereof and, usually, such compositions also contain a pharmaceutically acceptable carrier or diluent.

Pharmaceutical compositions comprising a compound of the present invention may be prepared by conventional techniques, e.g. as described in Remington: The Science and Practise of Pharmacy, 19^{th} Ed., 1995. The compositions may appear in conventional forms, for example capsules, tablets, aerosols, solutions or suspensions.

Typical compositions include a compound of the present invention or a pharmaceutically acceptable acid addition salt thereof, associated with a pharmaceutically acceptable excipient which may be a carrier or a diluent or be diluted by a carrier, or enclosed within a carrier which can be in form of a capsule, sachet, paper or other container. In making the compositions, conventional techniques for the preparation of pharmaceutical compositions may be used. For example, the active compound will usually be mixed with a carrier, or diluted by a carrier, or enclosed within a carrier, which may be in the form of a ampoule, capsule, sachet, paper, or other container. When the carrier serves as a diluent, it may be solid, semi-solid, or liquid material, which acts as a vehicle, excipient, or medium for the active compound. The active compound can be adsorbed on a granular solid container for example in a sachet. Some examples of suitable carriers are water, salt solutions, alcohols, polyethylene glycols, polyhydroxyethoxylated castor oil, syrup, peanut oil, olive oil, gelatine, lactose, terra alba, sucrose, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone.

The formulations may also include wetting agents, emulsifying and suspending agents, preserving agents, sweetening agents or flavouring agents.

The pharmaceutical preparations can be sterilized and mixed, if desired, with auxiliary agents, emulsifiers, salt for influencing osmotic pressure, buffers and/or coloring substances and the like, which do not deleteriously react with the active compounds.

The route of administration may be any route, which effectively transports the active compound to the appropriate or desired site of action, such as oral, nasal, pulmonary, transdermal or parenteral e.g. rectal, depot, subcutaneous, intramuscular or intranasal, the oral route being preferred.

If a solid carrier is used for oral administration, the preparation may be tabletted, placed in a hard gelatin capsule in powder or pellet form or it can be in the form of a troche or lozenge. If a liquid carrier is used, the preparation may be in the form of a syrup, emulsion, soft gelatin capsule or sterile injectable liquid such as an aqueous or non-aqueous liquid suspension or solution.

For nasal administration, the preparation may contain a compound of the present invention dissolved or suspended in a liquid carrier, in particular an aqueous carrier, for aerosol application. The carrier may contain additives such as solubilizing agents, e.g. propylene glycol, surfactants, absorption enhancers such as lecithin (phosphatidylcholine) or cyclodextrin, or preservatives such as parabenes.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

The compounds of the invention may be administered to a mammal, especially a human, in need of such reducing or lowering of the intake of fat food. Such mammals include also animals, both domestic animals, e.g. household pets, and non-domestic animals such as wildlife.

The compounds of the invention may be administered in the form of an alkali metal or earth alkali metal salt thereof, concurrently, simultaneously, or together with a pharmaceutically acceptable carrier or diluent, especially and preferably in the form of a pharmaceutical composition thereof, in an effective amount.

Pharmaceutical compositions containing a compound according to the invention may be administered one or more times per day or week, conveniently administered at mealtimes. An effective amount of such a pharmaceutical composition is the amount that provides a clinically significant effect against consumption of fat food. Such amounts will depend, in part, on the particular condition to be treated, age, weight, and general health of the patient, and other factors evident to those skilled in the art.

A convenient daily dosage can be in the range from 0.001-500 mg/kg/day. In another embodiment from 0.01-100 mg/kg/day. In a further embodiment from 0.05-50 mg/kg/day, and in yet another embodiment from 0.1-20 mg/kg/day. If the body weight of the subject changes during treatment, the dose of the compound might have to be adjusted accordingly.

Any novel feature or combination of features described herein is considered essential to this invention.

The present invention is further illustrated by the following example, which, however, are not to be construed as limiting the scope of protection. The features disclosed in the foregoing description and in the following examples may, both separately and in any combination thereof, be material for realising the invention in diverse forms thereof.

### EXAMPLES

### Example 1

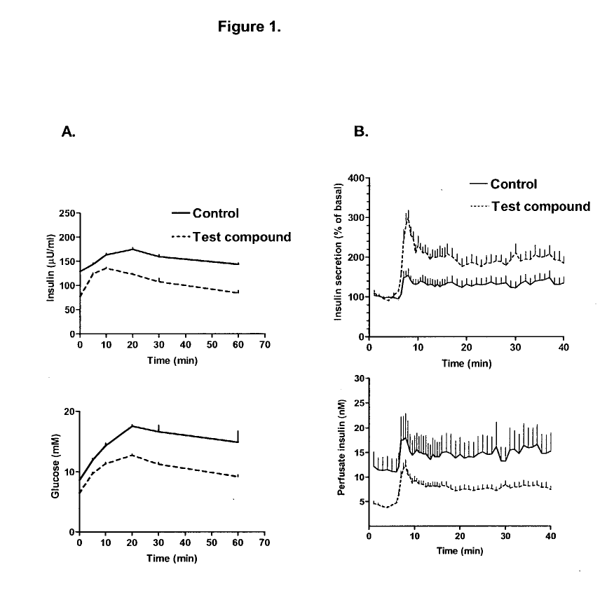

A study was performed in a colony of diabetic Zucker rats housed in Vancouver, Canada. This is a model of hyperinsulinemia and impaired glucose tolerance/mild type 2 diabetes. The aim was to investigate whether treatment with the test compound 6-chloro-3-(1-methylcylodopropyl)amino-4H-thieno[3,2-e]-1,2,4-thiadiazine 1,1-dioxide could lead to a reduction in hyperinsulinemia and an improvement in insulin secretory responsiveness to glucose in the perfused pancreas. Male diabetic Zucker rats were dosed for 3 weeks with either 1.5 mg/kg 6-chloro-3-(1-methylcylodopropyl)amino-4H-thieno[3,2-e]-1,2,4-thiadiazine 1,1-dioxide bid (n=8) or vehicle (n=16). Oral glucose tolerance was assessed in the animals the day after the last dose was given. In animals that had received 6-chloro-3-(1-methylcylodopropyl)amino-4H-thieno[3,2-e]-1,2,4-thiadiazine 1,1-dioxide, oral glucose tolerance was significantly improved and the improvement in glucose tolerance was associated with a significant reduction in hyperinsulinemia. Evaluation of perfused pancreas two days later revealed that pancreata from animals treated with 6-chloro-3-(1-methylcylodopropyl)amino-4H-thieno[3,2-e]-1,2,4-thiadiazine 1,1-dioxide displayed reduced hyperinsulinemia and an improved insulin secretory responsiveness to glucose when the data are expressed relative to baseline. The results are shown in FIG. 1.

The results demonstrate that it is possible to reduce hyperinsulinemia with 6-chloro-3-(1-methylcylodopropyl)amino-4H-thieno[3,2-e]-1,2,4-thiadiazine 1,1-dioxide without a deterioration of glucose tolerance and they furthermore demonstrate that the perfused pancreas is more responsive to a glucose challenge, analogously with the improved beta-cell compensation of for insulin resistance observed by Buchanan in women with former GDM treated with troglitazone. These features in combinations are claimed to be able to prevent or delay the onset of diabetes in women with prior GDM.

1. A use of SUR1/Kir6.2 selective potassium channel openers for the preparation of a pharmaceutical composition for the treatment or the prevention of diabetes in women with prior GDM.

2. A use of a compound of the general formula (I): 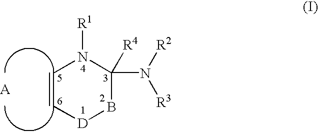
wherein
B represents >NR⁵ or >CR⁵R⁶, wherein R⁵ and R⁶ independently are hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen; or R⁵ and R⁴ together represent one of the bonds in a double bond between the atoms 2 and 3 of formula (I);
D represents —S(═O)₂— or —S(═O)—; or
D-B represents —S(═O)(R⁷)═N—
wherein R⁷ is C₁₋₆-alkyl; or aryl or heteroaryl optionally mono- or poly substituted with halogen, hydroxy, C₁₋₆-alkoxy, aryloxy, arylalkoxy, nitro, amino, C₁₋₆-monoalkyl- or dialkylamino, cyano, acyl, or C₁₋₆-alkoxycarbonyl;
R¹ is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen and R⁴ is hydrogen; or R⁴ together with R⁵ represent one of the bonds in a double bond between the atoms 2 and 3 of formula (I); or R¹ together with R⁴ represent one of the bonds in a double bond between the atoms 3 and 4 of formula (I);
R² is hydrogen; hydroxy; C₁₋₆-alkoxy; or C₁₋₆-alkyl, C₃₋₆-cycloalkyl, C₂₋₆-alkenyl or C₂₋₆-alkynyl optionally mono- or poly substituted with halogen;
R³ is R⁸; —OR⁸; —C(═X)R⁸; —NR⁸R⁹; bicycloalkyl, aryl, heteroaryl, arylalkyl or heteroarylalkyl optionally mono- or poly substituted with halogen, hydroxy, C₁₋₆-alkoxy, aryloxy, arylalkoxy, nitro, amino, C₁₋₆-monoalkyl- or dialkylamino, cyano, oxo, acyl or C₁₋₆-alkoxycarbonyl; or aryl substituted with C₁₋₆-alkyl;
wherein R⁸ is hydrogen; C₃₋₆-cycloalkyl or (C₃₋₆-cycloalkyl)C₁₋₆-alkyl, the C₃₋₆-cycloalkyl group optionally being mono- or poly substituted with C₁₋₆-alkyl, halogen, hydroxy or C₁₋₆-alkoxy; a 3-6 membered saturated ring system comprising one or more nitrogen, oxygen or sulfur atoms; or straight or branched C₁₋₁₈-alkyl optionally mono- or poly substituted with halogen, hydroxy, C₁₋₆-alkoxy, C₁₋₆-alkylthio, C₃₋₆-cycloalkyl, aryl, aryloxy, arylalkoxy, nitro, amino, C₁₋₆-monoalkyl- or dialkylamino, cyano, oxo, formyl, acyl, carboxy, C₁₋₆-alkoxycarbonyl, or carbamoyl;
X is O or S;
R⁹ is hydrogen; C₁₋₆-alkyl; C₂₋₆-alkenyl; C₃₋₆-cycloalkyl optionally mono- or poly substituted with C₁₋₆-alkyl, halogen, hydroxy or C₁₋₆-alkoxy; or
R⁸ and R⁹ together with the nitrogen atom form a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino, oxo; or
R³ is 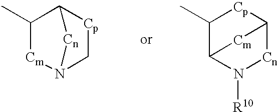
wherein n, m, p independently are 0, 1, 2, 3 and R¹⁰ is hydrogen; hydroxy; C₁₋₆-alkoxy; C₃₋₆-cycloalkyl optionally mono- or poly substituted with C₁₋₆-alkyl, halogen, hydroxy or C₁₋₆-alkoxy; C₁₋₆-alkyl, C₂₋₆-alkenyl or C₂₋₆-alkynyl optionally mono- or poly substituted with halogen; or
R² and R³ together with the nitrogen atom forms a 3-12 membered mono- or bicyclic system, in which one or more of the carbon atoms may be exchanged with nitrogen, oxygen or sulfur, each of these ring systems optionally being mono- or poly substituted with halogen, C₁₋₆-alkyl, hydroxy, C₁₋₆-alkoxy, C₁₋₆-alkoxy-C₁₋₆-alkyl, nitro, amino, cyano, trifluoromethyl, C₁₋₆-monoalkyl- or dialkylamino or oxo;
A together with carbon atoms 5 and 6 of formula (I) represents a 5 or 6 membered heterocyclic system comprising one or more nitrogen-, oxygen- or sulfur atoms, the heterocyclic systems optionally being mono- or poly substituted with halogen; C₁₋₁₂-alkyl; C₃₋₆-cycloalkyl; hydroxy; C₁₋₆-alkoxy; C₁₋₆-alkoxy-C₁₋₆-alkyl; nitro; amino; cyano; cyanomethyl; perhalomethyl; C₁₋₆-monoalkyl- or dialkylamino; sulfamoyl; C₁₋₆-alkylthio; C₁₋₆-alkylsulfonyl; C₁₋₆-alkylsulfinyl; C₁₋₆-alkylcarbonylamino; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl; C₁₋₆-alkoxycarbonyl-C₁₋₆-alkyl; carbamyl; carbamyl-methyl; C₁₋₆-monoalkyl- or dialkylaminocarbonyl; C₁₋₆-monoalkyl- or dialkylaminothiocarbonyl; ureido; C₁₋₆-monoalkyl- or dialkylaminocarbonylamino, thioureido; C₁₋₆-monoalkyl- or dialkylaminothiocarbonyl-amino; C₁₋₆-monoalkyl- or dialkylaminosulfonyl; carboxy; carboxy-C₁₋₆-alkyl; acyl; aryl, arylalkyl, aryloxy, the aryl group optionally being mono- or polysubstituted with C₁₋₆-alkyl, halogen, hydroxy or C₁₋₆-alkoxy; (1,2,4-oxadiazol-5-yl)- or (1,2,4-oxadiazol-3-yl)-C₁₋₆-alkyl the oxadiazolyl group optionally being substituted with C₁₋₆-alkyl or C₃₋₆-cycloalkyl; or a 5-6 membered nitrogen containing ring, optionally substituted with phenyl or C₁₋₆-alkyl; or a pharmaceutically acceptable salt thereof, for the preparation of a pharmaceutical composition for the treatment or the prevention of diabetes in women with prior Gestational Diabetes Mellitus (GDM).

3. The use according to claim 2 wherein B is >NR⁵ and R⁵ and R⁴ together represent one of the bonds in a double bond between the atoms 2 and 3 of formula (I).

4. The use according to claims 2 or 3 wherein D is —S(═O)₂−.

5. The use according to any of the claims 2-4 wherein R² is hydrogen or C₁₋₆-alkyl.

6. The use according to any of the claims 2-5 wherein R³ is R⁸, OR⁸, NR₃R⁹ or aryl, the aryl groups optionally being substituted with C₁₋₆-alkyl; wherein R⁸ is hydrogen; C₃₋₆-cycloalkyl; (C₃₋₆-cycloalkyl)C₁₋₆-alkyl; a 3-6 membered saturated ring system comprising one, two or three nitrogen, oxygen or sulfur atoms; or straight or branched C₁₋₁₈-alkyl optionally substituted with halogen, hydroxy, C₁₋₆-alkoxy, C₁₋₆-alkylthio, C₃₋₆-cycloalkyl or aryl; R⁹ is hydrogen, C₁₋₆-alkyl or C₃₋₆-cycloalkyl; or R⁸ and R⁹ together with the nitrogen R⁹ is hydrogen, C₁₋₆-alkyl or C₃₋₆-cycloalkyl; or R⁸ and R⁹ together with the nitrogen atom form a 4-6 membered ring.

7. The use according to any of the claims 2-6 wherein R³ is secondary C₃₋₆-alkyl, tertiary C₄₋₆-alkyl, C₃₋₆-cycloalkyl or (C₃₋₆-cycloalkyl)methyl.

8. The use according to any of the claims 2-7 wherein A together with carbon atoms 5 and 6 of formula (I) forms a 5 membered heterocyclic system containing one hetero atom selected from nitrogen and sulfur, the heterocyclic system optionally being mono- or disubstituted with halogen; C₁₋₁₂-alkyl; C₃₋₆-cycloalkyl; cyano; cyanomethyl; perhalomethyl; sulfamoyl; C₁₋₆-alkylthio; C₁₋₆-alkylsulfonyl; C₁₋₆-alkylsulfinyl; arylthio, arylsulfinyl, arylsulfonyl, the aryl group/optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl or a 5-6 membered nitrogen containing ring, optionally substituted with phenyl or C₁₋₆-alkyl.

9. The use according to any of the claims 2-8 wherein A together with carbon atoms 5 and 6 of formula (I) forms a 5 membered heterocyclic system containing two hetero atoms selected from nitrogen, oxygen and sulfur, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl; aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

10. The use according to any of the claims 2-9 wherein A together with carbon atoms 5 and 6 of formula (I) forms a 6 membered aromatic heterocyclic system containing one, two or three nitrogen atoms, the heterocyclic system optionally being substituted with halogen; C₁₋₁₂-alkyl; C₃₋₆-cycloalkyl; cyano; cyanomethyl; perhalomethyl; sulfamoyl; C₁₋₆-alkylthio; C₁₋₆-alkylsulfonyl; C₁₋₆-alkylsulfinyl; arylthio, arylsulfinyl, arylsulfonyl, the aryl group optionally being mono- or polysubstituted with C₁₋₆-alkyl, halogen, hydroxy or C₁₋₆-alkoxy; C₁₋₆-alkoxycarbonyl-C₁₋₆-alkyl; carbamylmethyl; carboxy-C₁₋₆-alkyl: aryloxy; (1,2,4-oxadiazol-5-yl)- or (1,2,4-oxadiazol-3-yl)C₁₋₆-alkyl, the oxadiazolyl group optionally being substituted with C₁₋₆-alkyl or C₃₋₆-cycloalkyl; acyl; or a 5-6 membered nitrogen containing ring, optionally substituted with phenyl or C₁₋₆-alkyl.

11. The use of a compound of the formula (I) according to any of the claims 2-10 selected from the group consisting of: 6-Chloro-3-(1,2-dimethylpropyl)amino-4H-thieno[3,2-e]-1,2,4-thiadiazine 1,1-dioxide; 6-Chloro-3-ethylamino-4H-thieno[3,2-e]-1,2,4-thiadiazine 1,1-dioxide; 6-Chloro-3-isopropylamino-4H-thieno[3,2-e]-1,2,4-thiadiazine 1,1-dioxide; (R)-6-Chloro-3-(1-phenylethyl)amino-4H-thieno[3,2-e]-1,2,4-thiadiazine 1,1-dioxide; 3-Allylamino-6-chloro-4H-thieno[3,2-e]-1,2,4-thiadiazine 1,1-dioxide; 6-Chloro-3-cyclopropylamino-4H-thieno[3,2-e]-1,2,4-thiadiazine 1,1-dioxide; 6-Chloro-3-hexylamino-4H-thieno[3,2-e]-1,2,4-thiadiazine 1,1-dioxide; 6-Chloro-3-tetradecylamino-4H-thieno[3,2-e]-1,2,4-thiadiazine 1,1-dioxide; 6-Chloro-3-methylamino-4H-thieno[3,2,e]-1,2,4-thiadiazine 1,1-dioxide; 3-Benzylamino-6-chloro-4H-thieno[3,2,e]-1,2,4-thiadiazine 1,1-dioxide; 6-Chloro-3-octylamino-4H-thieno[3,2-e]-1,2,4-thiadiazine 1,1-dioxide; 6-Chloro-3-isobutylamino-4H-thieno[3,2-e]-1,2,4-thiadiazine 1,1-dioxide; 6-Chloro-3-(4-phenylbutyl)amino-4H-thieno[3,2-e]-1,2,4-thiadiazine 1,1-dioxide; 6-Chloro-3-(1,5-dimethylhexyl)amino-4H-thieno[3,2-e]-1,2,4-thiadiazine 1,1-dioxide; 6-Chloro-3-propylamino-4H-thieno[3,2-e]-1,2,4-thiadiazine 1,1-dioxide; (R)-6-Chloro-3-(2-hydroxy-1-methylethyl)amino-4H-thieno[3,2-e]-1,2,4-thiadiazine 1,1-dioxide; (S)-6-Chloro-3-(2-hydroxy-1-methylethyl)amino-4H-thieno[3,2-e]-1,2,4-thiadiazine 1,1-dioxide; (R)-3-sec-Butylamino-6-chloro-4H-thieno[3,2-e]-1,2,4-thiadiazine 1,1-dioxide; 3-Butylamino-6-chloro-4H-thieno[3,2-e]-1,2,4-thiadiazine 1,1-dioxide; 3-Isopropylamino-7-methyl-4,7-dihydro-pyrazolo[4,3-e][1,2,4]thiadiazine 1,1-dioxide.

12. The use of a compound of the formula (I) according to any of the claims 2-10 selected from the group consisting of: 3-Hydrazino-4H-pyrido[4,3-e]-1,2,4-thiadiazine 1,1-dioxide; 3-Benzylamino-4H-pyrido[4,3-e]-1,2,4-thiadiazine 1,1-dioxide; 3-(R)-(1-Phenylethylamino)-4H-pyrido[4,3-e]-1,2,4-thiadiazine 1,1-dioxide; 3-(S)-(1-Phenylethylamino)-4H-pyrido[4,3-e]-1,2,4-thiadiazine 1,1-dioxide; 3-Benzylamino-7-chloro-4H-pyrido[2,3-e]-1,2,4-thiadiazine 1,1-dioxide; 7-Chloro-3-(R)-(1-phenylethylamino)-4H-pyrido[2,3-e]-1,2,4-thiadiazine 1,1-dioxide; 7-Chloro-3-(S)-(1′-phenylethylamino)-4H-pyrido[2,3-e]-1,2,4-thiadiazine 1,1-dioxide; 3-Benzylamino-4H-pyrido[2,3-e]-1,2,4-thiadiazine 1,1-dioxide; 3-(R)-(1-Phenylethylamino)-4H-pyrido[2,3-e]-1,2,4-thiadiazine 1,1-dioxide; 3-(S)-(1-Phenylethylamino)-4H-pyrido[2,3-e]-1,2,4-thiadiazine 1,1-dioxide; 3-(Hexylamino)-4H-pyrido[4,3-e]-1,2,4-thiadiazine 1,1-dioxide; 7-Chloro-3-hexylamino-4H-pyrido[2,3-e]-1,2,4-thiadiazine 1,1-dioxide; 3-Octylamino-4H-pyrido[4,3-e]-1,2,4-thiadiazine 1,1-dioxide; 7-Chloro-3-octylamino-4H-pyrido[2,3-e]-1,2,4-thiadiazine 1,1-dioxide; 3-Allylamino-4H-pyrido[4,3-e]-1,2,4-thiadiazine 1,1-dioxide; 3-Allylamino-7-chloro-4H-pyrido[2,3-e]-1,2,4-thiadiazine 1,1-dioxide; 7-Chloro-3-(2-methoxy-1-methylethyl)amino-4H-pyrido[2,3-e]-1,2,4-thiadiazine 1,1-dioxide; 3-(2-Methoxy-1-methylethyl)amino-4H-pyrido[4,3-e]-1,2,4-thiadiazine 1,1-dioxide; 3-(2-Hydroxy-1-methylethyl)amino-4H-pyrido[4,3-e]-1,2,4-thiadiazine 1,1-dioxide; 3-Benzylamino-2-methyl-2H-pyrido[4,3-e]-1,2,4-thiadiazine 1,1-dioxide; 2-Isopropylamino-3,3-dimethoxy-3H-pyrido[2,3-b][1,4]thiazine 4,4-dioxide.

13. The use of a compound of the formula (I) according to any of the claims 2-10 selected from the group consisting of: 7-Cyano-3-isopropylamino-6-methyl-4H-thieno[2,3-e]-1,2,4-thiadiazine 1,1-dioxide; 7-Cyano-6-methyl-3-propylamino-4H-thieno[2,3-e]-1,2,4-thiadiazine 1,1-dioxide; 6-Chloro-3-isopentylamino-4H-thieno[3,2-e]-1,2,4-thiadiazine 1,1-dioxide; 6-Chloro-3-(1-methylheptyl)amino-4H-thieno[3,2-e]-1,2,4-thiadiazine 1,1-dioxide; 6-Chloro-3-(1-ethylpentyl)amino-4H-thieno[3,2-e]-1,2,4-thiadiazine 1,1-dioxide; 6-Chloro-3-(2-methylbutyl) amino-4H-thieno[3,2-e]-1,2,4-thiadiazine 1,1-dioxide; 6-Chloro-3-(1-methylhexyl)amino-4H-thieno[3,2-e]-1,2,4-thiadiazine 1,1-dioxide; 6-Chloro-3-cyclopentylamino-4H-thieno[3,2-e]-1,2,4-thiadiazine 1,1-dioxide; 6-Chloro-3-cyclohexylmethylamino-4H-thieno[3,2-e]-1,2,4-thiadiazine 1,1-dioxide; Ethyl 3-(6-chloro-1,4-dihydro-1,1-dioxothieno[3,2-e]-1λ⁶,2,4-thiadiazin-3-ylamino)-butanoate; 3-(6-Chloro-1,4-dihydro-1,1-dioxothieno[3,2-e]-1λ⁶,2,4-thiadiazin-3-ylamino)butanoic acid; 6-Chloro-3-(3-hydroxy-1-methylpropyl)amino-4H-thieno[3,2-e]-1,2,4-thiadiazine 1,1-dioxide; (R)-6-Chloro-3-(1-phenyethyl)amino-4H-thieno[3,2-e]-1,2,4-thiadiazine 1,1-dioxide; (S)-3-sec-Butylamino-6-chloro-4H-thieno[3,2-e]-1,2,4-thiadiazine 1,1-dioxide; 6-Chloro-3-isopropylamino-4H-thieno[2,3-e]-1,2,4-thiadiazine 1,1-dioxide; 6-Chloro-3-cyclopentylamino-4H-thieno[2,3-e]-1,2,4-thiadiazine 1,1-dioxide; 6-Bromo-3-isopropylamino-4H-thieno[3,2-e]-1,2,4-thiadiazine 1,1-dioxide; 3-Isopropylamino-4H-thieno[3,2-e]-1,2,4-thiadiazine 1,1-dioxide; 6-Fluoro-3-isopropylamino-4H-thieno[3,2-e]-1,2,4-thiadiazine 1,1-dioxide; 3-Cyclobutylamino-5,6-dimethyl-4H-thieno[3,2-e]-1,2,4-thiadiazine 1,1-dioxide; 3-Cyclopentylamino-5,6-dimethyl-4H-thieno[3,2-e]-1,2,4-thiadiazine 1,1-dioxide; 3-Isopropylamino-6,7-dimethyl-4H-thieno[2,3-e]-1,2,4-thiadiazine 1,1-dioxide; 3-Cyclobutylamino-6,7-dimethyl-4H-thieno[2,3-e]-1,2,4-thiadiazine 1,1-dioxide; 3-Cyclopentylamino-6,7-dimethyl-4H-thieno[2,3-e]-1,2,4-thiadiazine 1,1-dioxide; 5-Chloro-3-isopropylamino-4H-thieno[3,2-e]-1,2,4-thiadiazine 1,1-dioxide; 5-Chloro-3-propylamino-4H-thieno[3,2-e]-1,2,4-thiadiazine 1,1-dioxide; 5-Chloro-3-cyclopentylamino-4H-thieno[3,2-e]-1,2,4-thiadiazine 1,1-dioxide; 5-Chloro-6-methyl-3-isopropylamino-4H-thieno[3,2-e]-1,2,4-thiadiazine 1,1-dioxide; 6-Chloro-3-isopropylamino-5-methyl-4H-thieno[3,2-e]-1,2,4-thiadiazine 1,1-dioxide; 6-Chloro-3-cyclopentylamino-5-methyl-4H-thieno[3,2-e]-1,2,4-thiadiazine 1,1-dioxide; 6-Fluoro-3-propylamino-4H-thieno[3,2-e]-1,2,4-thiadiazine 1,1-dioxide; 6-Fluoro-3-cyclopentylamino-4H-thieno[3,2-e]-1,2,4-thiadiazine 1,1-dioxide; 5-Fluoro-3-propylamino-4H-thieno[3,2-e]-1,2,4-thiadiazine 1,1-dioxide; 5-Fluoro-3-isopropylamino-4H-thieno[3,2-e]-1,2,4-thiadiazine 1,1-dioxide; 3-Isopropylamino-7-methyl-4H-thieno[2,3-e]-1,2,4-thiadiazine 1,1-dioxide; 6-Chloro-3-cyclobutylamino-4H-thieno[3,2-e]-1,2,4-thiadiazine 1,1-dioxide; 6-Chloro-3-(2-hydroxyethyl)amino-4H-thieno[3,2-e]-1,2,4-thiadiazine 1,1-dioxide; (+)-3-exo-Bicyclo[2.2.1]hept-2-ylamino-6-chloro-4H-thieno[3,2-e]-1,2,4-thiadiazine 1,1-dioxide;
(R)-6-Chloro-3-(2-hydroxypropyl)amino-4H-thieno[3,2-e]-1,2,4-thiadiazine 1,1-dioxide; 6-Bromo-3-isopropylamino-4H-thieno[3,2-e]-1,2,4-thiadiazine 1,1-dioxide; 5,6-Dibromo-3-isopropylamino-4H-thieno[3,2-e]-1,2,4-thiadiazine 1,1-dioxide; 6-Chloro-3-cyclohexylamino-4H-thieno[3,2-e]-1,2,4-thiadiazine 1,1-dioxide; 6-Chloro-3-(furan-2-ylmethyl)amino-4H-thieno[3,2-e]-1,2,4-thiadiazine 1,1-dioxide; 6-Chloro-3-(1-ethylpropyl) amino-4H-thieno[3,2-e]-1,2,4-thiadiazine 1,1-dioxide; 6-Bromo-3-cyclopentylamino-4H-thieno[3,2-e]-1,2,4-thiadiazine 1,1-dioxide; 6-Chloro-3-(2-methylallyl)amino-4H-thieno[3,2-e]-1,2,4-thiadiazine 1,1-dioxide; 6-Cyano-3-isopropylamino-4H-thieno[3,2-e]-1,2,4-thiadiazine 1,1-dioxide.

14. The use of a compound of the formula (I) according to claim 2 having the general formula (Ia): 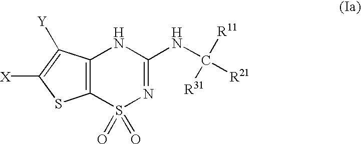
wherein
X and Y independently are hydrogen, halogen, perhalomethyl, C₁₋₆-alkyl or C₁₋₆-alkoxy;
R¹¹, R²¹ and R³¹ independently are C₁₋₆-alkyl, C₂₋₆-alkenyl, C₂₋₆-alkynyl, C₃₋₆-cycloalkyl, carboxy, C₁₋₆-alkoxycarbonyl or aryl, all of which are optionally being mono- or polysubstituted with halogen, hydroxy, oxo, or aryl; or
R¹¹ is as defined above and R²¹—C—R³¹ form a C₃₋₆-cycloalkyl group, optionally being mono- or polysubstituted with C₁₋₆-alkyl, perhalomethyl, halogen, hydroxy or aryl; or
—CR¹¹R²¹R³¹ form a 4- to 12-membered bicyclic or tricyclic carbocyclic system, optionally being mono- or polysubstituted with C₁₋₆-alkyl, perhalomethyl, halogen, hydroxy or aryl; or a salt thereof with a pharmaceutically acceptable acid or base including all optical isomers of compounds of formula (Ia) for the preparation of a pharmaceutical composition for the prevention or the treatment of diabetes in women with prior Gestational Diabetes Mellitus (GDM).

15. The use of a compound according to claim 14 wherein X is halogen and Y is hydrogen.

16. The use of a compound according to claims 14 or 15 wherein in formula (Ia), X is chloro.

17. The use of a compound according to any of the claims 14-16 wherein in formula (Ia), R¹¹, R²¹ and R³¹ all are C₁₋₆-alkyl.

18. The use of a compound according to any of the claims 14-17 wherein in formula (Ia), R¹¹ is methyl.

19. The use of a compound according to any of the claims 14-18 wherein in formula (Ia), R²¹—C—R³¹ forms a C₃₋₆-cycloalkyl group.

20. The use of a compound according to any of the claims 14-19 wherein in formula (Ia), —CR¹¹R²¹R³¹ forms a tricyclic carbocyclic system.

21. The use of a compound according to any of the claims 14-20 selected from the group consisting of 3-tert-butylamino-6-chloro-4H-thieno[3,2-e]-1,2,4-thiadiazine 1,1-dioxide; 6-Chloro-3-(1,1-dimethylpropylamino)-4H-thieno[3,2-e]-1,2,4-thiadiazine 1,1-dioxide; 6-Chloro-3-(1-methylcyclopropyl)amino-4H-thieno[3,2-e]-1,2,4-thiadiazine 1,1-dioxide; 6-Chloro-3-(2-hydroxy-1,1-dimethylethylamino)-4H-thieno[3,2-e]-1,2,4-thiadiazine 1,1-dioxide; 6-Chloro-3-(1,1,3,3-tetramethylbutylamino)-4H-thieno[3,2-e]-1,2,4-thiadiazine 1,1-dioxide; 3-(1-Adamantyl)amino-6-chloro-4H-thieno[3,2-e]-1,2,4-thiadiazine 1,1-dioxide; 1-(6-Chloro-1,4-dihydro-1, 1-dioxo-thieno[3,2-e]-1λ⁶,2,4-thiadiazin-3-ylamino)-cyclopropanecarboxylic acid ethyl ester; 6-Chloro-3-(1-methyl-1-phenylethyl)amino-4H-thieno[3,2-e]-1,2,4-thiadiazine 1,1-dioxide; 6-Chloro-3-(1-hydroxymethylcyclopentyl)amino-4H-thieno[3,2-e]-1,2,4-thiadiazine 1,1-dioxide; 1-(6-Chloro-1,4-dihydro-1,1-dioxo-thieno[3,2-e]-1λ⁶,2,4-thiadiazin-3-ylamino)-cyclopropanecarboxylic acid; 6-Chloro-3-(1-methylcyclobutyl)amino-4H-thieno[3,2-e]-1,2,4-thiadiazine 1,1-dioxide; 6-Chloro-3-(1-methylcyclohexyl)amino-4H-thieno[3,2-e]-1,2,4-thiadiazine 1,1-dioxide; 6-Chloro-3-(1-methylcyclopentyl)amino-4H-thieno[3,2-e]-1, 2,4-thiadiazine 1,1-dioxide; 6-Chloro-3-(1-ethylcyclobutyl)amino-4H-thieno[3,2-e]-1,2,4-thiadiazine 1,1-dioxide.

22. The use of a compound according to any of the claims 14-21 which is 6-chloro-3-(1-methylcyclopropyl)amino-4H-thieno[3,2-e]-1,2,4-thiadiazine 1,1-dioxide.

23. A pharmaceutical composition for use in the treatment or prevention of diabetes in women with prior GDM, comprising a compound of the formula (I) or (Ia) or a pharmaceutically acceptable salt thereof with a pharmaceutically acceptable acid or base, or any optical isomer or mixture of optical isomers including a racemic mixture, or any tautomeric form together with one or more pharmaceutically acceptable carriers or diluents.

24. The pharmaceutical composition according to claim 23 in the form of an oral dosage unit or parental dosage unit.

25. The pharmaceutical composition according to claim 23 wherein said compound of the formula (I) or (Ia) is administered as a dose in the range from about 0.001 to 500 mg/kg/day, particularly from about 0.01 to 100 mg/kg/day and especially in the range from 0.05 to 50 mg/kg/day.

26. A method for treating or preventing diabetes in women with prior GDM comprising administering an effective amount of a compound of the formula (I) or (Ia) to said subject.
